(19) United States
(12) Patent Application Publication
(10) Pub. No.: US 2025/0241661 A1
(43) Pub. Date: Jul. 31, 2025

(54) Vertebral Disc Preparation Instrument With an Articulated Scoop Assembly

(71) Applicant: Life Spine, Inc., Huntley, IL (US)

(72) Inventor: Daniel P. Predick, Wheat Ridge, CO (US)

(73) Assignee: Life Spine, Inc., Huntley, IL (US)

(21) Appl. No.: 19/040,703

(22) Filed: Jan. 29, 2025

Related U.S. Application Data
(60) Provisional application No. 63/626,756, filed on Jan. 30, 2024.

(51) Int. Cl. A61B 17/16; A61B 17/00

(52) U.S. Cl. CPC A61B 17/1671; A61B 2017/00469

(57) ABSTRACT

A medical instrument for collecting spinal disc material from outside a perimeter of a tube has a handle, a shaft assembly defining a proximal end extending from the handle, a distal end, and a longitudinal axis, and an articulated scoop assembly at the distal end of the shaft assembly that provides for swiveling movement of a scoop of the scoop assembly relative to the longitudinal axis of the shaft assembly to provide movement of the scoop beyond the perimeter of the tube. The shaft assembly includes a controller coupled to and actuated by the handle and configured to manipulate swiveling of the scoop. The handle is formed by a handgrip portion configured to receive a palm of a user and a lever portion with a loop configured to receive one or more fingers of the user. The handle is biased so the scoop is normally coaxial with the shaft assembly.

### CROSS-REFERENCE TO RELATED APPLICATIONS

This U.S. non-provisional patent application claims the benefit of and/or priority under 35 U.S.C. § 119 (e) to U.S. provisional patent application Ser. No. 63/626,756 filed Jan. 30, 2024 titled “Vertebral Disc Preparation Instrument With an Articulated Scoop Assembly,” the entire contents of which is specifically incorporated herein by reference.

### FIELD OF THE INVENTION

The present invention relates to instruments for spine procedures and, more particularly, to disc preparation instruments.

### BACKGROUND OF THE INVENTION

Many people contend with spine issues due to age, disease, trauma, congenital, and acquired complications and conditions. While some spine issues can be alleviated without surgery, other spine issues necessitate surgery. In the case of problems with a vertebral disc of the spine, it may be necessary to insert an implant in the vertebral disc space between adjacent vertebrae. Before the implant is inserted into the vertebral disc space, the vertebral disc or disc material (hereinafter, disc material) in the vertebral disc space must first be removed.

This spine procedure is now commonly accomplished using minimally invasive surgery (MIS) or microsurgery. Both procedures reduce trauma by using surgical instruments that are introduced into the body via a separate cannula that has been inserted into the body through a small incision and positioned accordingly. A medical instrument for removing vertebral disc material is then inserted into the cannula. The end of the medical instrument is configured to acquire and hold vertebral disc material and/or tissue for removal from the body. The medical instrument is then extracted from the cannula where the acquired disc material and/or tissue can be removed from the configured end.

Heretofore, most configured end of medical instruments for obtaining and removing vertebral disc material have either been fixed in co-axial orientation relative to a longitudinal axis of the elongated shaft of the medical instrument or have limited movement outside of the longitudinal axis of the elongated shaft. This limits the reach of the configured end and thus the ability of the configured end to effectively obtain more disc and/or tissue from a single position of the cannula.

It would therefore be advantageous to have a medical instrument for removing vertebral disc material and tissue via a cannula that can extend beyond the longitudinal axis of the cannula and the end medical instrument. It would furthermore be advantageous to have an articulating medical instrument that provides better access to the vertebral disc space, greater removal of vertebral disc material, greater degree of sweep, and extension beyond the longitudinal axis of the cannula and the medical instrument.

The present medical instrument addresses the above and more.

### SUMMARY OF THE INVENTION

A medical instrument for use with a tube is configured to be inserted through the tube and collect spinal disc material from outside the perimeter of the tube via an articulated scoop assembly at a distal end of the medical instrument.

The medical instrument has a handle, a shaft assembly defining a proximal end extending from the handle, a distal end, and a longitudinal axis, and an articulated scoop assembly at the distal end of the shaft assembly that provides for swiveling movement of a scoop of the articulated scoop assembly relative to the longitudinal axis of the shaft assembly to allow movement of the scoop beyond the perimeter of the tube. The shaft assembly includes a controller coupled to and actuated by the handle and configured to influence the articulated scoop assembly and thus swiveling of the scoop.

The controller of the shaft assembly is situated for axial movement relative to the shaft assembly through manipulation of the handle whereby the articulated scoop assembly causes the scoop to swivel from a position coaxial with the longitudinal axis of the shaft assembly through a pivot arc that extends beyond a perimeter of the longitudinal axis of the shaft assembly and an associated tube, and back.

The shaft assembly may have an upper shaft and a lower shaft with the controller situated in the upper shaft. A lower rod may then be connected to the controller distal end, with the controller including a longitudinal slot proximate its distal end that is coaxial with the longitudinal axis of the shaft assembly that retains a first pin situated at the distal end of the lower rod of the shaft assembly such that the controller is constrained to longitudinal movement relative to an upper rod of the shaft assembly. The scoop has a second pin at its proximal end that is constrained to perpendicular movement relative to the longitudinal axis of the shaft assembly via a transverse slot at the distal end of the lower rod. Back and forth longitudinal movement of the lower rod moves the second pin of the scoop up and down within the transverse slot creating a pivot that swivels the scoop. Each side of the scoop has an arced slot that rides respectfully in third and fourth pins at the distal end of the lower rod thereby allowing the scoop to swivel up and down relative to the longitudinal axis of the shaft assembly and thus beyond the perimeter of the shaft assembly and any associated tube, and back to a position coaxial with the longitudinal axis of the shaft assembly.

The handle is formed by a handgrip portion configured to receive a palm of a user and a lever portion with a finger loop configured to receive one or more fingers of the user. A double leaf spring is situated on and between the handgrip portion and the lever portion to provide tension between the handgrip portion and the lever portion when the handgrip portion and the lever portion are squeezed together during use, and to automatically return the lever portion to an initial state when squeezing of the handgrip portion and the lever portion has ceased. The handgrip portion may include a thumb hold.

Further aspects of the present invention will become apparent from consideration of the drawings and the following description of a form of the invention. A person skilled in the art will realize that other forms of the invention are possible and that the details of the invention can be modified in a number of respects without departing from the inventive concept. The following drawings and description are to be regarded as illustrative in nature and not restrictive.

### BRIEF DESCRIPTION OF THE DRAWINGS

The present invention and its features will be better understood by reference to the accompanying drawings, wherein:

FIG. 1 is a view of a medical instrument fashioned in accordance with the present principles for removing vertebral disc material from a vertebral disc space between adjacent vertebrae of a spine using a cannula during spine surgery;

FIG. 2 is an enlarged view of a scoop assembly at the distal end of a shaft assembly of the medical instrument of FIG. 1 with a scoop of the scoop assembly shown in an un-articulated position;

FIG. 3 is another enlarged view of the scoop assembly at the distal end of the shaft assembly of the medical instrument of FIG. 1 with the scoop of the scoop assembly shown in an un-articulated position;

FIG. 4 is an enlarged sectional view of the scoop assembly at the distal end of the shaft assembly of the medical instrument of FIG. 1 taken along line 4-4 of FIG. 3;

FIG. 5 is an enlarged view of the scoop assembly at the distal end of the shaft assembly of the medical instrument of FIG. 1 with the scoop of the scoop assembly shown in an articulated position; and

FIG. 6 is an enlarged sectional view of the scoop assembly at the distal end of the shaft assembly of the medical instrument of FIG. 1 taken along line 6-6 of FIG. 5.

For the purpose of promoting an understanding of the principles of the invention, reference will now be made to the embodiment illustrated in the drawings and specific language will be used to describe the same. It will nevertheless be understood that no limitation of the scope of the invention is thereby intended. Any alterations and further modifications in the described embodiment, and any further applications of the principles of the invention as described herein are contemplated as would normally occur to one skilled in the art to which the invention relates.

### DETAILED DESCRIPTION OF THE INVENTION

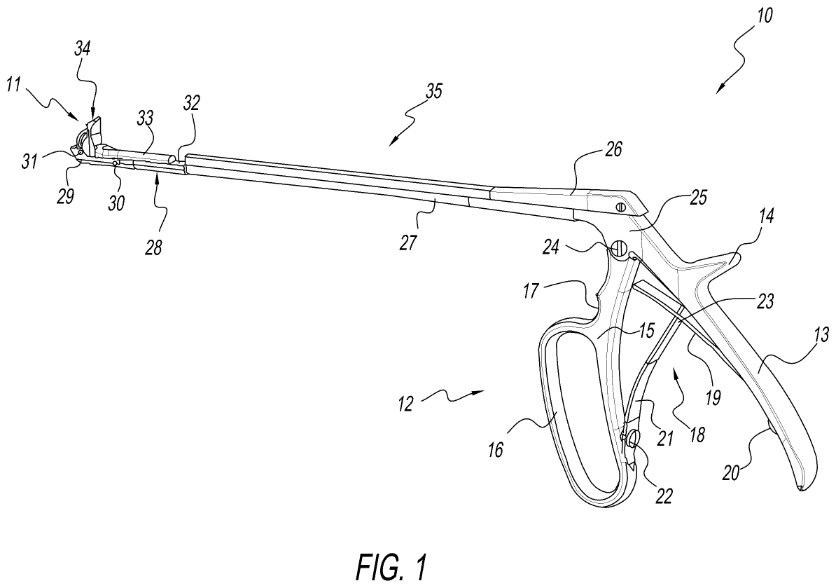

Referring to FIGS. 1-6, there is shown a medical instrument, generally designated 10, for obtaining and removing vertebral disc material and tissue (not shown), collectively vertebral disc material, from a vertebral disc space (not shown) of a spine (not shown) during a surgical spine procedure (surgical spine procedure), particularly, but not necessarily, using a tube (not shown but well known in the art). The medical instrument 10 is sized for use in the tube and is made from one or more surgical grade materials. With reference to FIG. 1, the medical instrument 10 has a user-graspable handle or handle assembly (handle) 12 and a shaft assembly 35 extending from the handle 12 and terminating at distal end 11. The handle 12 includes a first leg 13 shaped to be placed in a palm (not shown) of a hand (not shown) of an operator (not shown) with a projection 14 sized and situated to receive a thumb (not shown) of the operator's hand. The handle 12 further includes a second leg 15 connected to an upper portion 25 of the first leg 13 via a pivot 24. The nomenclature first and second being arbitrary here and throughout unless specially specified otherwise. The second leg 15 has a loop 16 sized and configured to receive the lower fingers (not shown) of the operator's hand, and a notch 17 between the loop 16 and the upper portion 25 sized and configured to receive a forefinger (not shown) of the operator's hand. The handle 12 is thus configured to allow an operator to grasp, hold and operate the medical instrument 10.

Situated between the first leg 13 of the handle 12 and the second leg 15 of the handle is a spring device fashioned as a double leaf spring 18 or similar device preferably, but not necessarily, made of metal and defined by a first elongated spring portion 19 attached by a fastener 20 to the inside of the first leg 13, and a second elongated spring portion 21 attached by a fastener 22 to the inside of the second leg 15. The unattached end of the first elongated spring portion 19 and the unattached end of the second elongated spring portion 21 meet and connect at juncture 23 forming the leaf spring 18. The handle 12 is configured to be normally in an open position as depicted in FIG. 1. The shaft assembly 35 is defined by first elongated arm or shaft 26 connected to and extending from a top area of the upper portion 25 of the first leg 13 of the handle 12, and a second elongated arm or shaft 27 connected to and extending from a bottom area of the upper portion of the first leg 13, the second elongated shaft 27 positioned below the first elongated shaft 26.

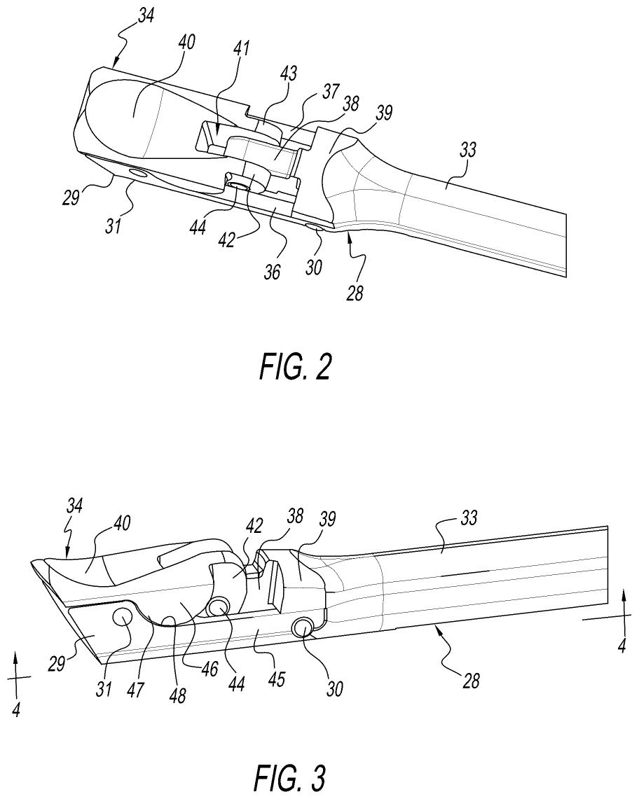

An extension 28 extends axially from the distal end of the second elongated shaft 27 which ends in a distal forked body 45 defining a first branch 36 and a second branch 37 (sec, e.g., FIG. 2). A rod 32 is housed within the first elongated shaft 26 and is connected at its proximal end to the second leg 15. Application of pressure to (e.g. squeezing of) the second leg 15 of the handle 12 towards the first leg 13 of the handle 12 against tension of the double leaf spring 18 pivots the second leg 15 and thus axially moves the rod 32 within the first shaft 26 coaxial with a longitudinal axis LA (see FIG. 5) of the shaft assembly 35. The rod 32 has a broad portion 33 at its distal end that is sized such that its proximal end can abut the distal end of the first elongated shaft 26 when the handle 12 is in a normal position and thus the rod 32 is in a fully axially rearward position. The broad portion 33 of the rod 32 has an outwardly flared neck 39 at its distal end.

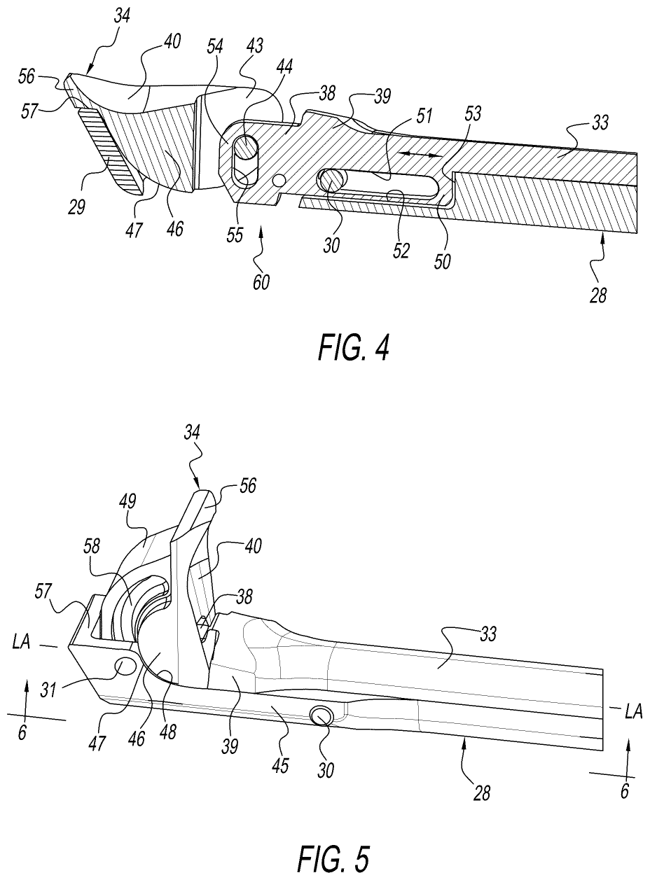

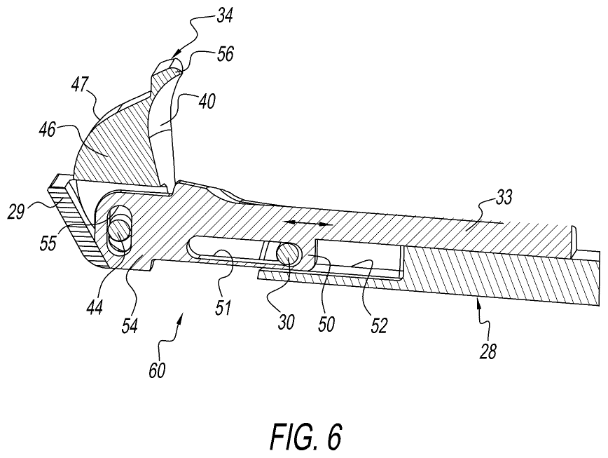

As best seen in FIG. 4, the distal extension 28 has a longitudinal or horizontal groove 52 defining a back or vertical wall 53. The broad portion 33 has a lower elongated flange 50 that is received in the longitudinal groove 52 for longitudinal movement of the lower elongated flange 50 therein and longitudinal movement of the rod 32. The lower elongated flange 50 has a longitudinal or horizontal slot 51. A first pin 30 is situated at the distal end of the distal extension 28 transverse to the longitudinal axis LA of the shaft assembly 35 and is received in the longitudinal slot 51 to constrain the broad portion 33 and the rod 32 to longitudinal movement along the longitudinal axis LA. Distal to the neck 39 is a reduction section 38 having a distal flange 54. The distal flange 54 has a slot 55 transverse to the longitudinal axis LA of the shaft assembly 35. A second pin 44 at a proximal end of a scoop 34 having a concavity 40 is received in the transverse slot 55 and is thus constrained to transverse movement therein. These form an articulated scoop assembly 60 having a scoop 34. Longitudinal movement of the first elongated shaft 26 in a forward direction (to the left in the figures) translates the second pin 44 downward (see, e.g., FIG. 6) causing the scoop 34 to pivot upward to a position beyond the perimeter of the shaft assembly 35 and any associated tube. Longitudinal movement of the first elongated shaft 26 in a rearward direction (to the right in the figures) translates the second pin 44 upward (see, e.g., FIG. 6) causing the scoop 34 to pivot downward to a position coaxial with the longitudinal axis LA of the shaft assembly 35.

The scoop 34 has a main body 46 with a curved end 43 at its distal end with a bifurcation defining a first boss 42 and a second boss 43 that hold the second pin 44. The second pin 44 also extends through a distal end 38 of the broad portion 33. The main body 46 has a generally curved lower surface 47 that abuts an oppositely curved surface 48 of the distal end of the forked body 45 such that the curved lower surface 47 rides along the oppositely curved surface 48 when the scoop 34 is caused to pivot upwards and downwards. The distal end of the forked body 45 has an upward extending flange 29 having an inside surface that creates a stop against which a surface 49 of the main body 46 abuts when the scoop 34 is forced forward by the forward movement of the rod 32 in the first elongated shaft 26. The scoop 34 further has a first curved groove 58 on one lateral side and a second curved groove (not seen) in a second lateral side opposite the first lateral side. A third pin 31 is provided at a first transverse distal side of the forked body 45 that extends into the first curved groove 58. A fourth pin (not seen) is provided at a second transverse distal side (not seen) of the forked body 45 that extends into the second curved groove (not seen). These structures help guide the scoop upwards and downwards. Variations may be used.

When the rod 32 is caused to move forward by manipulation of the handle 12 (overcoming the bias of the double leaf spring 18), the scoop 34 is pushed forward as the second pin 44 rides downward in the transverse slot 55 which pushes the curved surface 49 against the inside surface of the non-moving upward extending flange 29 thereby pushing the scoop 34 upwards beyond the perimeter of the shaft assembly 35 and any associated tube. Release of the pressure on the handle causes the double leaf spring 18 to bias the handle 12 back to its normal position, causing the rod 32 to move rearward. This pulls the second pin 44 upwards in the transverse slot 55 which pulls the scoop 34 back into the position coaxial with the longitudinal axis of the shaft assembly 35.

While the invention has been illustrated and described in detail in the drawings and foregoing description, the same is to be considered as illustrative and not restrictive in character, it being understood that only a preferred embodiment has been shown and described and that all changes and modifications that come within the spirit of the invention are desired to be protected. It should be understood that while the use of words such as preferable, preferably, preferred or more preferred utilized in the description above indicate that the feature so described may be more desirable, it nonetheless may not be necessary and embodiments lacking the same may be contemplated as within the scope of the invention.

What is claimed is:

1. A medical instrument configured for insertion through a tube placed in a body and the collection of bodily tissue, the medical instrument comprising:
a user-graspable handle;
a shaft assembly extending from the user-graspable handle and defining a longitudinal axis, and an end distal to the user-graspable handle;
an articulated scoop assembly having a scoop at the distal end of the shaft assembly and configured to provide swiveling movement of the scoop relative to the longitudinal axis of the shaft assembly whereby the scoop has an arc of swivel that extends beyond a perimeter of the shaft assembly; and
a controller associated with the user-graspable handle and the shaft assembly and configured to be actuated by the user-graspable handle to control swiveling of the scoop.

2. A medical instrument comprising:
a user-graspable handle;
a shaft assembly defining a proximal end extending from the user-graspable handle, a distal end, and a longitudinal axis;
an articulated scoop assembly at the distal end of the shaft assembly configured for swiveling movement of a scoop of the scoop assembly relative to the longitudinal axis of the shaft assembly to allow movement of the scoop beyond the perimeter of the shaft assembly; and
a controller associated with the shaft assembly and coupled to and actuated by the user-graspable handle and configured to influence swiveling of the scoop, the controller defining a distal controller end.

3. The medical instrument of claim 2, wherein the controller of the shaft assembly is situated for axial movement relative to the shaft assembly through manipulation of the user-graspable handle whereby the articulated scoop assembly causes the scoop to swivel from a position coaxial with the longitudinal axis of the shaft assembly through a pivot arc that extends beyond a perimeter of the longitudinal axis of the shaft assembly, and back.

4. The medical instrument of claim 3, wherein:
the shaft assembly includes an upper shaft and a lower shaft;
the controller includes a longitudinal slot proximate the controller distal end that is coaxial with the longitudinal axis of the shaft assembly and that retains a first pin situated at the distal end of a lower rod of the shaft assembly such that the controller is constrained to longitudinal movement relative to an upper rod of the shaft assembly;
the scoop has a second pin at its proximal end that is constrained to perpendicular movement relative to the longitudinal axis of the shaft assembly via a transverse slot at the distal end of the lower rod wherein back and forth longitudinal movement of the lower rod moves the second pin of the scoop up and down within the transverse slot creating a pivot that swivels the scoop; and
each side of the scoop has an arced slot that rides respectfully in third and fourth pins at the distal end of the lower rod thereby allowing the scoop to swivel up and down relative to the longitudinal axis of the shaft assembly and thus beyond the perimeter of the shaft assembly and any associated tube, and back to a position coaxial with the longitudinal axis of the shaft assembly.

5. The medical instrument of claim 4, wherein the user-graspable handle is formed by a handgrip portion configured to receive a palm of a user and a lever portion with a finger loop configured to receive one or more fingers of the user; and
further comprising a double leaf spring situated on and between the handgrip portion and the lever portion to provide tension between the handgrip portion and the lever portion when the handgrip portion and the lever portion are squeezed together during use, and to automatically return the lever portion to an initial state when squeezing of the handgrip portion and the lever portion has ceased.

6. The medical instrument of claim 5, wherein the handgrip portion includes a thumb hold.

7. A method of collecting spinal disc material from a vertebral disc of a spine comprising:
providing a tube;
placing the tube in a body of a patient proximate a spinal disc;
providing a medical instrument configured for be inserted through the tube and collect spinal disc material from outside the perimeter of the tube, the medical instrument comprising:
a user-graspable handle;
a shaft assembly defining a proximal end extending from the user-graspable handle, a distal end, and a longitudinal axis;
an articulated scoop assembly at the distal end of the shaft assembly configured for swiveling movement of a scoop of the scoop assembly relative to the longitudinal axis of the shaft assembly to allow movement of the scoop beyond the perimeter of the shaft assembly; and
a controller associated with the shaft assembly and coupled to and actuated by the user-graspable handle and configured to influence swiveling of the scoop, the controller defining a distal controller end;
manipulating the user-graspable handle of the medical instrument to cause the controller to cause the articulated scoop assembly to pivot the scoop beyond a perimeter of the tube and collect spinal disc material;
collecting spinal disc material from the spinal disc via the scoop;
manipulating the user-graspable handle of the medical instrument to cause the controller to cause the articulated scoop assembly to pivot the scoop with collected spinal disc material into a position wherein the scoop is coaxial with the longitudinal axis of the shaft assembly; and
removing the medical instrument from the tube.

8. The method of claim 7, wherein the controller of the shaft assembly is situated for axial movement relative to the shaft assembly through manipulation of the user-graspable handle whereby the articulated scoop assembly causes the scoop to swivel from a position coaxial with the longitudinal axis of the shaft assembly through a pivot arc that extends beyond a perimeter of the longitudinal axis of the shaft assembly, and back.

9. The method of claim 8, wherein:
the shaft assembly includes an upper shaft and a lower shaft;
the controller includes a longitudinal slot proximate the controller distal end that is coaxial with the longitudinal axis of the shaft assembly and that retains a first pin situated at the distal end of a lower rod of the shaft assembly such that the controller is constrained to longitudinal movement relative to an upper rod of the shaft assembly;
the scoop has a second pin at its proximal end that is constrained to perpendicular movement relative to the longitudinal axis of the shaft assembly via a transverse slot at the distal end of the lower rod wherein back and forth longitudinal movement of the lower rod moves the second pin of the scoop up and down within the transverse slot creating a pivot that swivels the scoop; and
each side of the scoop has an arced slot that rides respectfully in third and fourth pins at the distal end of the lower rod thereby allowing the scoop to swivel up and down relative to the longitudinal axis of the shaft assembly and thus beyond the perimeter of the shaft assembly and any associated tube, and back to a position coaxial with the longitudinal axis of the shaft assembly.

10. The method of claim 9, wherein the user-graspable handle is formed by a handgrip portion configured to receive a palm of a user and a lever portion with a finger loop configured to receive one or more fingers of the user; and
further comprising a double leaf spring situated on and between the handgrip portion and the lever portion to provide tension between the handgrip portion and the lever portion when the handgrip portion and the lever portion are squeezed together during use, and to automatically return the lever portion to an initial state when squeezing of the handgrip portion and the lever portion has ceased.

11. The method of claim 10, wherein the handgrip portion includes a thumb hold.
